(19) United States
(12) Patent Application Publication
(10) Pub. No.: US 2021/0093223 A1
(43) Pub. Date: Apr. 1, 2021

(54) CYLINDRICAL BODY HAVING A THREE-AXIS MAGNETIC SENSOR

(71) Applicant: KING ABDULLAH UNIVERSITY OF SCIENCE AND TECHNOLOGY, Thuwal (SA)

(72) Inventors: Jürgen KOSEL, Thuwal (SA); Mayyada HAWSAWI, Thuwal (SA); Abdullah Saud ALMANSOURI, Thuwal (SA)

(21) Appl. No.: 17/041,748

(22) PCT Filed: Apr. 5, 2019
(86) PCT No.: PCT/IB2019/052819

§ 371 Date: Sep. 25, 2020

Related U.S. Application Data
(60) Provisional application No. 62/653,861, filed on Apr. 6, 2018.

(51) Int. Cl. A61B 5/06; A61B 5/00

(52) U.S. Cl. CPC A61B 5/062; A61B 5/6852; A61B 2562/164; A61B 2562/0223; A61B 5/742

(57) ABSTRACT

A device includes a cylindrical body having a proximate end and a distal end. A three-axis magnetic sensor is mounted on the proximate end of the cylindrical body. The three-axis magnetic sensor includes an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body.

### CROSS-REFERENCE TO RELATED APPLICATIONS

This application claims priority to U.S. Provisional Patent Application No. 62/653,861, filed on Apr. 6, 2018, entitled “METHOD FOR ORIENTATION TRACKING OF CARDIAC CATHETERS,” the disclosure of which is incorporated herein by reference in its entirety.

### BACKGROUND

### Technical Field

Embodiments of the subject matter disclosed herein generally relate to a cylindrical body having a three-axis magnetic sensor that can detect an orientation of the cylindrical body using the Earth's magnetic field.

### Discussion of the Background

Modern surgery methods utilize minimally invasive tools and robotic assistance to greatly increase the efficiency of medical treatments while reducing costs and risks for the patients, patient discomfort, as well as hospitalization times. For example, cardiac catheterization is a minimally invasive surgery that is performed to diagnose or treat cardiovascular conditions. During surgery, a catheter is threaded to the heart from a vein in the groin, neck, or arm. The catheter helps the cardiac surgeon in diagnosing the heart by locating any blockage in the blood vessels, obtaining a tissue sample from the heart or checking the pumping function of the heart. The catheter can also be used for treating the heart during procedures such as angioplasty, closure of holes, replacement of heart valves, and ablation.

The common method for visualizing the heart during the surgery uses fluoroscopy, which requires the use of a contrast agents and x-ray imaging in order to confirm the position and orientation of the catheter tip, as well as check the blood flow in the coronary arteries. Thus, a continuous x-ray beam must be passed through the body of the patient to provide the surgeon with a visual image of the heart and the catheter that are presented on a display.

While cardiac catheterization is minimally invasive and generally considered safe compared to open surgery, there exist serious side effects that may affect the patients. One of the major limitations is the use of x-ray and contrast agents for determining the position and orientation of the catheter. This typically involves a series of attempts to get into the targeted vessel branch and put the catheter into the intended position and orientation, leading to a series of contrast dye injections and extended x-ray exposure. It should be noted that one of the common causes of acute renal dysfunction is the contrast medium-induced nephropathy (CIN), which has gained increased attention in clinical settings, especially during cardiac intervention. The occurrence of CIN is reported to be 2% in the general population; however, the percentage could rise up to 20%-30% in high-risk patients (i.e., patients with chronic renal impairment, diabetes mellitus, congestive heart failure, and older age). Higher contrast volume is a serious risk factor for CIN. Another concern is the long x-ray exposure, which causes radiation side effects to both the patients and medical staff.

A recently developed technology that aims to decrease the x-ray exposure during cardiac catheterization involves a remote magnetic navigation system consisting of two focused-field permanent magnets inside of a housing that are positioned on each side of the patient's body. The two magnets create a relatively uniform magnetic field of approximately 0.08 T, which can penetrate 15 cm inside the chest of the patient. A small permanent magnet is placed on the tip of the catheter, which aligns itself with the applied magnetic field produced by the magnets outside of the body. If there is a change in the orientations of the outer magnets with respect to each other, the magnetic field changes accordingly and as a consequence the catheter tip gets deflected. Thereby, the surgeon can navigate the catheter via a computer-controlled system without any manual manipulation. However, this method requires special equipment and installation, making it a bulky and a very expensive solution. In addition, it is not compatible with the force measurement tools that are currently being used in hospitals and does not provide position or orientation feedback.

A three-axis magneto-impedance sensor system has also been proposed as a navigation tool to detect the position and orientation of a catheter tip to minimize x-ray exposure during cardiac catheterization. The system exploits the Earth magnetic field together with an AC magnetic field of 10 kHz that is produced by a two-axis magnetic field coil. A 3-axis magnetoimpedance effect sensor is used to detect both the Earth magnetic field and the AC magnetic field. Two Euler angles are obtained from the measurements of the Earth magnetic field while the AC magnetic field measurements provide the third Euler angle, representing the orientation of the tip. Even though this system offers a navigation system that potentially minimizes x-ray exposure, it requires the detection of two different magnetic fields, making the system complicated. Further, although magnetoimpedance sensors provide high sensitivity, these sensors operate at high frequencies (around 100 MHz to GHz), which requires complex electronics, and which makes them very sensitive to changes in their environment (i.e., different tissues types etc.). Most significantly, magneto-impedance sensors are relatively bulky. The size of the sensor used was 2 mm×2 mm×3 mm, which is very large compared to sizes of catheter devices, and there is little potential for further miniaturization.

Thus, there is a need to be able to determine the orientation of a catheter, or other cylindrical body, that minimizes the use of contrast agents and x-rays and is small enough that it does not impact the use of the catheter and is compatible with additional measurement tools.

### SUMMARY

According to an embodiment, there is a device, which includes a cylindrical body having a proximate end and a distal end. A three-axis magnetic sensor is mounted on the proximate end of the cylindrical body. The three-axis magnetic sensor includes an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body.

According to another embodiment, there is a method, which involves detecting a change in an orientation of a cylindrical body having a proximate end and a distal end. A three-axis magnetic sensor is mounted on the proximate end of the cylindrical body. The three-axis magnetic sensor includes an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body. An updated orientation of the cylindrical body is determined using the detected change in orientation. The change in orientation is detected using the Earth's magnetic field and without inducing an external magnetic field.

According to a further embodiment, there is a method, which involves providing a cylindrical body having a proximate end and a distal end and mounting a three-axis magnetic sensor on the proximate end of the cylindrical body. The three-axis magnetic sensor includes an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1A is a schematic diagram of a device according to embodiments;

FIGS. 1B and 1C are schematic diagrams of a side and top view, respectively, of a device according to embodiments;

FIG. 2 is a schematic diagram of a device according to embodiments;

FIG. 3 is a schematic diagram of a device according to embodiments;

FIG. 4 is a schematic diagram of a magnetic tunnel junction sensor according to embodiments;

FIG. 5 is a flowchart of a method of using a device according to embodiments; and

FIG. 6 is a flowchart of a method of forming a device according to embodiments.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of a catheter. However, the embodiments are applicable to any type of cylindrical body, such as an endoscope, flexible inspection cameras, drillers, etc.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

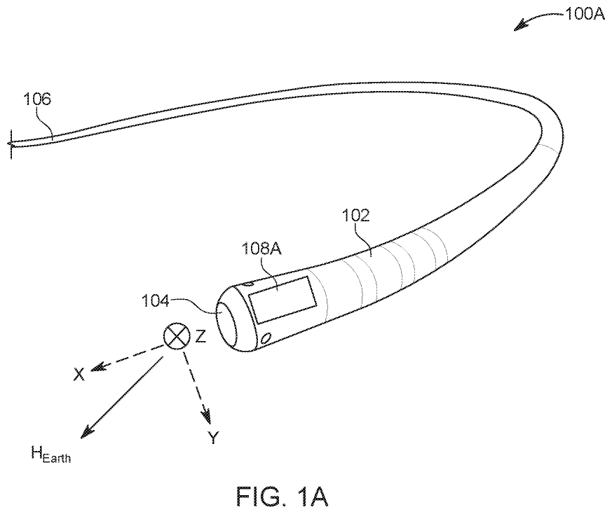

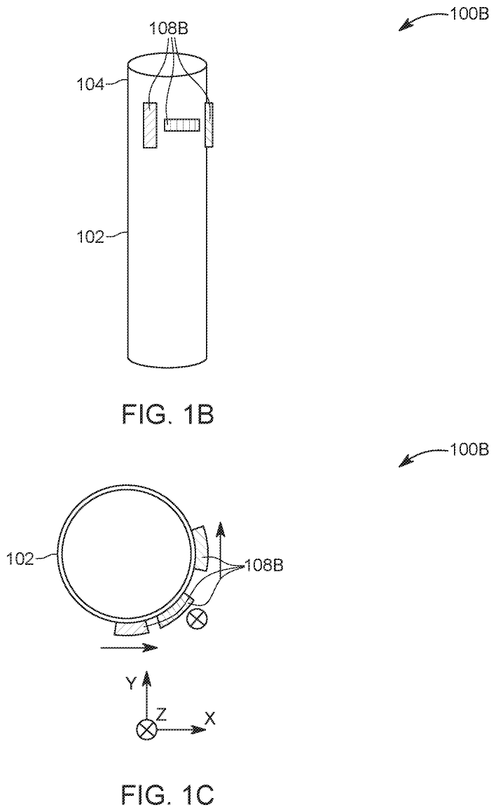

FIG. 1A is a schematic diagram of a device according to embodiments. The device 100A includes a cylindrical body 102 having a proximate end 104 and a distal end 106 and a three-axis magnetic sensor 108A mounted on the proximate end 104 of the cylindrical body 102. As used herein, the term cylindrical body refers to a body having a generally cylindrical form even if the body does not satisfy the mathematical definition of a cylinder. The three-axis magnetic sensor includes three magnetic sensors, an X-axis magnetic sensor sensitive to magnetic fields along an X-axis of the cylindrical body 102, a Y-axis magnetic sensor sensitive to magnetic fields along a Y-axis of the cylindrical body 102, and a Z-axis magnetic sensor sensitive to magnetic fields along a Z-axis of the cylindrical body 102. This involves orienting the three magnetic sensors so that they are arranged on the cylindrical body 102 with a proper orientation to detect the directional changes. Specifically, the X-axis magnetic sensor is arranged orthogonal to the Y-axis magnetic sensor and the Z-axis magnetic sensor is arranged orthogonal to the X-axis and Y-axis magnetic sensors. The X-, Y-, and Z-axis magnetic sensors can be magnetic tunnel junction sensors, giant magnetoresistance sensors, Hall effect sensors, etc. Regardless of the type of magnetic sensor, the X-, Y-, and Z-axis magnetic sensors are flexible enough so that each magnetic sensor conforms to the shape of the cylindrical body 102.

In the embodiment illustrated in FIG. 1A, the X-, Y-, and Z-axis magnetic sensors are arranged on a common flexible substrate, which could be, for example, a flexible silicon or flexible polyimide substrate. Accordingly, it is helpful to know how the three magnetic sensors are arranged on the common substrate when the three-axis magnetic sensor 108A is mounted on the cylindrical body 102 so that the signals provided by the different magnetic sensors can be correlated with directional changes in the different axes. Alternatively, which ones of the magnetic sensors are oriented to detect changes can be performed after the three-axis magnetic sensor 108A is mounted by separately adjusting the orientation of the cylindrical body in at least two of the axes and determining which one of the magnetic sensors is detecting directional changes.

FIGS. 1B and 1C are schematic diagrams of a side and top view, respectively, of a device according to embodiments. This embodiment is similar to the one illustrated in FIG. 1A except that in this embodiment the three-axis magnetic sensor 108B is comprised of three magnetic sensors (i.e., X-, Y-, and Z-axis sensors) formed on separate flexible substrates. Otherwise, the embodiment illustrated in FIG. 1A is identical to the embodiment illustrated in FIGS. 1B and 1C and both operate in the same manner.

The three-axis magnetic sensor employs the Earth's magnetic field for detecting the orientation without requiring additional external magnetic fields. Specifically, each of the three magnetic sensors acts as a variable resistor controlled by the Earth's impinging magnetic field. Thus, depending on the orientation of the proximate end 104 of the cylindrical body 102, the resistivity of the three magnetic sensors will be changed by the Earth's magnetic field. Accordingly, the resistance values of each of the magnetic sensors correlate to different orientations of the proximate end 104 of the cylindrical body 102.

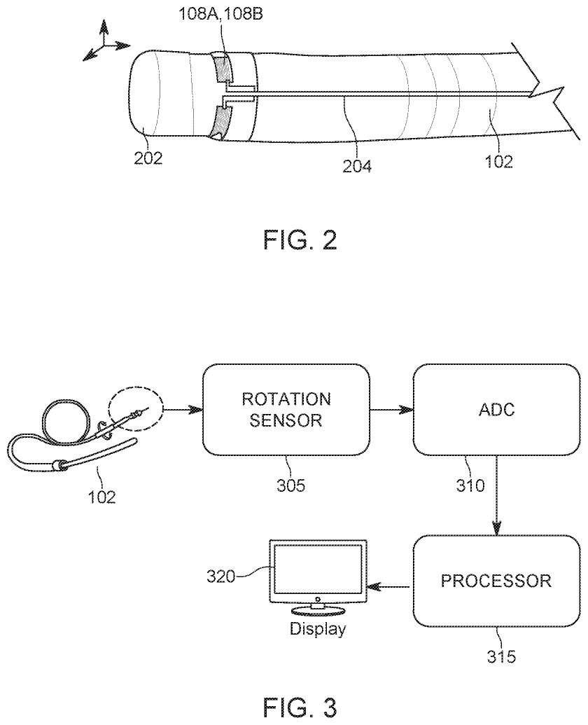

FIG. 2 is a schematic diagram of a device according to embodiments. In the illustrated embodiment, the three-axis magnetic sensor 108A or 108B is arranged beneath a protective cap 202. Further, the cylindrical body 102 includes electrodes 204, which electrically couple the three-axis magnetic sensor 108A or 108B to a rotation sensor (not illustrated), which is located on the distal end of the cylindrical body 102. Although the electrodes 204 are illustrated as being arranged on an outer surface of the cylindrical body 102, the electrodes 204 can be arranged beneath a protective covering on the outside of the cylindrical body 102. Further, although not illustrated in FIG. 2, additional electrical wiring can be provided on the cylindrical body to power the three-axis magnetic sensor 108A or 108B. Because magnetic sensors are relatively low power devices, the amount of power traveling along the cylindrical body 102 is so small that it does not present any danger to a patient in which a portion of the cylindrical body is inserted.

FIG. 3 is a schematic diagram of a device according to embodiments. As illustrated, the cylindrical body 102, which carries the three-axis magnetic sensor 108A or 108B on its proximate end, includes a rotation sensor 305 on the distal end of the cylindrical body 102. The rotation sensor 305 converts changes in resistivity of one or more of the X-, Y-, and Z-axis magnetic sensors into analog values corresponding to an amount of displacement of the X-, Y-, and Z-axis magnetic sensors about their corresponding axis. The rotation sensor 305 is coupled to an analog-to-digital (ADC) converter 310, which converts the analog values into digital and provides the digital values to processor 315. Processor 315 uses these digital values to represent the current orientation of the cylindrical body 102 on display 320. Thus, an operator of this device is provided with visual feedback on the current orientation of the cylindrical body 102 about the X-, Y-, and Z-axes.

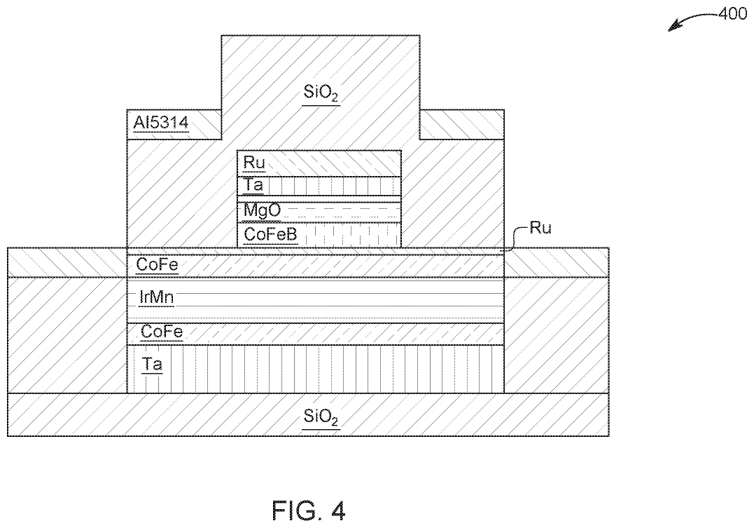

FIG. 4 is a schematic diagram of a magnetic tunnel junction sensor according to embodiments, which can form the X-, Y-, and Z-axis magnetic sensors. The use of a magnetic tunnel junction sensor as the X-, Y-, and Z-axis magnetic sensors is presented as an example of one type of magnetic sensor and the discussion of this example should not be considered as limiting the disclosed magnetic sensors to being only magnetic tunnel junction sensors. Instead, the disclosed magnetic sensors can alternatively be giant magnetoresistance sensors, Hall effect sensors, etc.

The illustrated magnetic tunnel junction sensor 400 includes a magnesium oxide (MgO) tunnel barrier and a cobalt iron boron (CoFeB) free layers. It has been found that the tunnel magnetoresistance (TMR) and hysteresis change rapidly around a critical free layer thickness of 15 Å, with a linear and hysteresis-free response when the thickness of the of the CoFeB free layer is less than this critical value. This behavior is attributable to a transition in the CoFeB free layer from the original ferromagnetic state to a superparamagnetic state as the thickness is reduced. The magnetic tunnel junction sensor 400 has the following structure (thicknesses in angstroms):

Substrate/Ta_50/Ru_300/Ta_50/Co 70 Fe 30_20/IrMn_150/Co 70 Fe 30_20/Ru_8/Co 40 Fe 20 B 20_30/MgO_20.5/Co 40 Fe 40 B 20_1 6/contact layer

The magnetic tunnel junction sensor 400 can achieve a TMR ratio greater than 150% for linear response and a sensitivity, in terms of resistance change, of 4.93%/Oe. The magnetic tunnel junction sensor 400 has a low power consumption of 0.15 μW, which minimizes heating at the tip of the catheter, and thus minimizes any impact on surrounding tissue during surgery. The magnetic tunnel junction sensor has volume of 150 μm², a height of 5 μm, and weighs 8 μg. Further, the magnetic tunnel junction sensor 400 can be bent up to 500 μm without impairing its functionality, and thus can be arranged on even the smallest catheters currently in use having a diameter of 1 mm (3F). The magnetic tunnel junction sensor 400 can be formed on a conventional silicon oxide substrate and after the device is formed, the silicon oxide substrate can be back-etched to make the device flexible. For example, the magnetic tunnel junction sensor 400 can be formed on a 500 μm silicon substrate, which is only 5 μm thick after the back etching.

It should be recognized that the magnetic tunnel junction 400 illustrated in FIG. 4 is one example of a magnetic tunnel junction that can be used, and it should be recognized that the disclosed embodiments can employ magnetic tunnel junctions having different structures and/or dimensions than that illustrated in FIG. 4. Further, as noted above, the X-, Y-, and Z-axis magnetic sensors can also be giant magnetoresistance sensors, Hall effect sensors, etc.

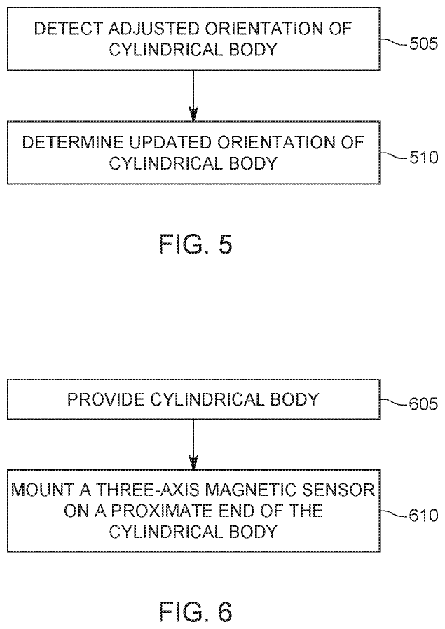

FIG. 5 is a flowchart of a method of using a device according to embodiments. Initially, a change in an orientation of a cylindrical body 102 having a proximate end 104 and a distal end 106 is detected (step 505). A three-axis magnetic sensor 108A or 108B is mounted on the proximate end 104 of the cylindrical body 102. The three-axis magnetic sensor 108A or 1086 comprises an X-axis magnetic sensor sensitive to magnetic fields along an X-axis of the cylindrical body 102, a Y-axis magnetic sensor sensitive to magnetic fields along a Y-axis of the cylindrical body 102, and a Z-axis magnetic sensor sensitive to magnetic fields along a Z-axis of the cylindrical body 102. An updated orientation of the cylindrical body 102 is determined using the detected change in orientation (step 510). As discussed above, the change in orientation is detected using the Earth's magnetic field and without inducing an external magnetic field. Further, as also discussed above, the adjusted orientation is detected based on changes in electrical resistance of the three-axis magnetic sensor 108A or 108B. Specifically, the adjusted orientation is detected based on changes in electrical resistance of the X-, Y-, and Z-axis magnetic sensors. The method can include additional steps, such as, outputting the determined updated orientation of the cylindrical body to a display.

FIG. 6 is a flowchart of a method of forming a device according to embodiments. Initially, a cylindrical body 102 having a proximate end 104 and a distal end 106 is provided (step 605). A three-axis magnetic sensor 108A or 1086 is then mounted on the proximate end 104 of the cylindrical body 102 (step 610). The three-axis magnetic sensor 108A or 108B comprises an X-axis magnetic sensor sensitive to magnetic fields along an X-axis of the cylindrical body 102, a Y-axis magnetic sensor sensitive to magnetic fields along a Y-axis of the cylindrical body 102, and a Z-axis magnetic sensor sensitive to magnetic fields along a Z-axis of the cylindrical body 102. The mounting of the three-axis magnetic sensor can involve mounting the X-axis magnetic sensor on a first flexible substrate in a first orientation, mounting the Y-axis magnetic sensor on a second flexible substrate in a second orientation, and mounting the Z-axis magnetic sensor on a third flexible substrate in a third orientation. Alternatively, the X-, Y-, and Z-axis magnetic sensors can be mounted on a common flexible substrate.

The method can include additional steps, including electrically coupling the three-axis magnetic sensor to a rotation sensor, electrically coupling a processor to the rotation sensor, and/or electrically coupling a display to the processor to display an orientation of the cylindrical body.

The disclosed device with a three-axis magnetic sensor provides a number of advantages over conventional techniques for determining the orientation of a cylindrical body, such as a catheter. Specifically, the device has a simple construction because it relies upon the Earth's magnetic field and does not require an external magnetic field. This simple design allows for the three-axis magnetic sensor to be arranged on the proximate end of the catheter without impairing the intended use of the catheter. Further, the processing electronics, such as the rotation sensor, are arranged on the distal end of the catheter, thereby not taking up valuable space on the proximate end of the catheter. By providing a simple way for determining the orientation of the catheter, the use of dyes and x-ray radiation required by conventional techniques can be minimized.

The disclosed embodiments provide a cylindrical body with a three-axis magnetic sensor. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A device, comprising:
a cylindrical body having a proximate end and a distal end; and
a three-axis magnetic sensor mounted on the proximate end of the cylindrical body, wherein the three-axis magnetic sensor comprises an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body.

2. The device of claim 1, wherein the three-axis magnetic sensor comprises:
the X-axis magnetic sensor on a first flexible substrate;
the Y-axis magnetic sensor on a second flexible substrate; and
the Z-axis magnetic sensor on a third flexible substrate.

3. The device of claim 1, wherein the three-axis magnetic sensor comprises:
the X-axis magnetic sensor, the Y-axis magnetic sensor, and the Z-axis magnetic sensor all mounted on a common flexible substrate.

4. The device of claim 1, wherein the X-axis magnetic sensor, the Y-axis magnetic sensor, and the Z-axis magnetic sensor are magnetic tunnel junction sensors, giant magnetoresistance sensors, or Hall effect sensors.

5. The device of claim 1, wherein the three-axis magnetic sensor is on a flexible silicon substrate or flexible polyimide substrate.

6. The device of claim 5, wherein the three-axis magnetic sensor conforms to a shape of the cylindrical body.

7. The device of claim 1, further comprising:
a rotation sensor electrically coupled to the three-axis magnetic sensor, wherein the rotation sensor is mounted on the distal end of the cylindrical body.

8. The device of claim 7, further comprising:
a processor coupled to the rotation sensor; and
an output coupled to the processor, wherein the processor provides the output with a current rotation of the cylindrical body.

9. The device of claim 1, wherein the device is a catheter.

10. The device of claim 1, wherein the device is an endoscope.

11. A method comprising:
detecting a change in an orientation of a cylindrical body having a proximate end and a distal end, wherein a three-axis magnetic sensor is mounted on the proximate end of the cylindrical body, and the three-axis magnetic sensor comprises an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body; and
determining an updated orientation of the cylindrical body using the detected change in orientation.

12. The method of claim 11, wherein the change in orientation is detected using the Earth's magnetic field.

13. The method of claim 12, wherein the change in orientation is detected without inducing an external magnetic field.

14. The method of claim 11, wherein the adjusted orientation is detected based on changes in electrical resistance of the X-, Y-, and Z-axis magnetic sensors.

15. The method of claim 11, further comprising:
outputting the determined updated orientation of the cylindrical body to a display.

16. A method, comprising:
providing a cylindrical body having a proximate end and a distal end; and
mounting a three-axis magnetic sensor on the proximate end of the cylindrical body, wherein the three-axis magnetic sensor comprises an X-axis magnetic sensor sensitive to magnetic fields along an X-axis of the cylindrical body, a Y-axis magnetic sensor sensitive to magnetic fields along a Y-axis of the cylindrical body, and a Z-axis magnetic sensor sensitive to magnetic fields along a Z-axis of the cylindrical body.

17. The method of claim 16, wherein the mounting of the three-axis magnetic sensor comprises:
mounting the X-axis magnetic sensor on a first flexible substrate in a first orientation;
mounting the Y-axis magnetic sensor on a second flexible substrate in a second orientation; and
mounting the Z-axis magnetic sensor on a third flexible substrate in a third orientation.

18. The method of claim 16, further comprising:
electrically coupling the three-axis magnetic sensor to a rotation sensor.

19. The method of claim 18, further comprising:
electrically coupling a processor to the rotation sensor.

20. The method of claim 19, further comprising:
electrically coupling a display to the processor to display an orientation of the cylindrical body.
